(19) United States
(12) Patent Application Publication
(10) Pub. No.: US 2026/0315359 A1
(43) Pub. Date: Oct. 15, 2026

(54) TOPICAL COMPOSITIONS AND METHODS FOR REVERSING DRUG EFFECTS TO SKIN

(71) Applicant: Aforé LLC, Libertyville, IL (US)

(72) Inventors: Julius Few, Chicago, IL (US); Alec Semersky, Libertyville, IL (US)

(21) Appl. No.: 19/649,007

(22) Filed: Apr. 15, 2026

Related U.S. Application Data
(60) Provisional application No. 63/789,418, filed on Apr. 15, 2025.

(51) Int. Cl. A61K 8/67; A61K 8/64; A61K 8/73; A61Q 19/00

(52) U.S. Cl. CPC A61K 8/671; A61K 8/64; A61K 8/735; A61Q 19/00; A61K 2800/28

(57) ABSTRACT

Topical compositions include a retinoid in an aqueous medium. The compositions can also comprise an aliphatic oligopeptide, an acetylated oligopeptide, and/or hyaluronic acid. Methods of administering topical compositions include topically and/or subtopically administering to facial skin of a subject.

### CROSS-REFERENCE TO RELATED APPLICATIONS

This application claims the benefit of U.S. Provisional Application No. 63/789,418, filed Apr. 15, 2025, the contents of which are incorporated by reference in their entirety.

### BACKGROUND

Glucagon-like-peptide-1 receptor agonists (GLP-1 RAs) are a popular class of drugs that are Food and Drug Administration approved to treat type 2 diabetes as well as obesity and cardiovascular disease prevention [1]. As more GLP-1 RAs come to market with decreased prices, simpler use, and at-home availability via telehealth providers, the percentage of GLP-1 RA treated patients in aesthetic plastic surgical settings is likely to increase. Though the safety profile of GLP-1 RAs is favorable, the drugs do have side effects recognized in the medical literature, including gastrointestinal distress, pancreatic concerns, cardiovascular effects, and injection site reactions [2]. Some investigators are beginning to look at the effects of GLP-1 RA on adipose and skeletal muscle.

Glucocorticoids and systemic steroids serve as an effective treatment for hyperproliferative skin conditions, such as hypertrophic scarring, by helping to suppress inflammation, and reduce keratosis [7]. By suppressing keratinocyte proliferation and migration, these treatments can effectively manage certain inflammatory skin conditions that cause excess keratosis, like psoriasis [8]. Glucocorticoid administration for variable term are shown to inhibit keratinocyte and fibroblast activity at the cellular and cytokine level [9, 10]. These changes have lead to increased skin susceptibility to ultraviolet damage, decreased elasticity, decreased barrier function, increased transepidermal water loss, and increased fragility [11, 12].

Use of GLP-1 RAs may result in similar skin changes to those observed in patients on systemic and glucocorticoids, due to similar antiproliferative effects on keratinocytes, macrophages, and fibroblasts. Just as glucocorticoids and systemic steroids reduce keratinocyte proliferation, so too do GLP-1 RAs [9][6]. Keratinocytes have been found to exhibit GLP1 receptors, and their proliferation is modulated by GLP-1 RAs—Liraglutide specifically [6]. In studies examining liraglutide's effects on keratinocytes, it was found both in vitro and in vivo that liraglutide suppressed keratinocyte proliferation [6][5]. Similar to glucocorticoids, liraglutide has been proposed as a potential treatment for psoriasis, given its anti-proliferative effects [4].

### SUMMARY

As one aspect of the present disclosure, methods are provided for improving one or more facial aesthetics of a subject. The methods comprise applying to facial skin of a subject one of the topical compositions described herein, wherein the composition is applied at least once per day for at least 21 days, alternatively for at least 42 days. In some embodiments, the composition is applied to the facial skin without removal.

As another aspect of the present disclosure, topical compositions are provided. The compositions comprise a retinoid, an aliphatic oligopeptide, and hyaluronic acid in an aqueous medium. The retinoid can be retinol, retinal, a retinyl ester, or a mixture thereof. The aliphatic oligopeptide can be a palmitoyl oligopeptide such as palmitoyl tetrapeptide-7 or palmitoyl tripeptide-1. In some embodiments, components through are present in the particular amounts or concentrations disclosed herein. The present compositions can be formulated as serums, creams, lotions, oils, salves, liniments, ointments, gels, pastes, or facial masks.

These and other features and advantages of the present methods and compositions will be apparent from the following detailed description, in conjunction with the appended claims.

### BRIEF DESCRIPTION OF THE DRAWINGS

FIG. 1 shows components of the Aesthetic Global Ranking Scale (GRS) used for evaluating an embodiment of the present topical composition in Example 2.

FIG. 2 shows mean GRS scores from Example 2 at baseline, day 21, and day 42 for left and right sides of the face.

FIGS. 3, 4, and 5 are photographs of subjects treated in Example 2.

FIG. 6 illustrates various components of an embodiment of the present composition and their expected functions.

The present teachings are best understood from the following detailed description when read with the accompanying figures. The features are not necessarily drawn to scale. Wherever practical, like reference numerals refer to like features.

### DETAILED DESCRIPTION

The present formulation contains bioavailable peptide and vitamin A derivatives that increase keratinocyte proliferation in the skin, stimulate elastin and collagen production in the skin, improve skin barrier function, and reduce visible signs of aging via topical application to the skin. Based on anecdotal findings in facelift surgery, we hypothesize that there is a direct link between GLP-1 treatment and dermal/epidermal impairment. In vitro and in vivo studies have demonstrated GLP-1 RAs suppressing keratinocyte proliferation, suppressing cytokine production, reducing skin structural integrity and barrier function, and reducing epidermal thickness [4][5][6]. We theorize a major source of pathology for what we refer to as “GLP-1 Face” is the result of dermal impairment [3]. To our knowledge, the present disclosure represents the first time an analogy between glucocorticosteroid and GLP-1 RA effect on the physiology of the skin, thus identifying an important consequence of GLP-1 RA treatment and potential ways to combat it.

Taken together with the observed accelerated aging, and textural differences observed during facelift surgery on GLP-1 RA treated patients, we hypothesize that long term use of GLP-1 RAs may lead to skin atrophy through a similar mechanism to glucocorticoids and systemic steroids. In the burn literature, it is known that patients with large surface area burns, on systemic steroid therapy, that large dose systemic vitamin A partially reverses steroid induced transforming growth factor beta 1 and insulin like growth factor 1 suppression, thus protecting the dermis and epidermis from classic steroid changes [13].

It is to be understood that the terminology used herein is for purposes of describing particular embodiments only, and is not intended to be limiting. The defined terms are in addition to the technical and scientific meanings of the defined terms as commonly understood and accepted in the technical field of the present teachings.

### Methods Of Applying To And Treating Facial Skin

As one aspect, the present disclosure provides methods of improving one or more facial aesthetics of a subject receiving or about to receive a therapeutic having an adverse effect on one or more fascial aesthetics. The method comprises applying to facial skin of the identified subject a topical composition comprising an agent that reverses or ameliorates the adverse effect of the therapeutic on said facial aesthetics. Examples of therapeutics having an adverse effect on one or more fascial aesthetics include GLP-1 agonists, SGLT-2 inhibitors, corticosteroids, or any condition that is associated with premature thinning of the skin.

In some embodiments, the subject is identified as receiving or about to receive a therapeutic having an adverse effect on one or more facial aesthetics by the subject's disclosure to a physician or healthcare provider. Alternatively, the subject is identified based on medical records, prescription records or health insurance claims.

In some embodiments, the therapeutic having an adverse effect on one or more fascial aesthetics is a GLP-1 receptor agonist. Examples of GLP-1 receptor agonists include semaglutide (marketed as Ozempic and Wegovy), liraglutide (marketed as Victoza), dulaglutide (marketed as Trulicity), exenatide (marketed as Byetta), exenatide extended-release (marketed as Bydureon), lixisenatide (marketed as Adlyxin), oral semaglutide (marketed as Rybelsus), and tirzepatide (marketed as Mounjaro and Zepbound). As used herein, the term “GLP-1 receptor agonist” means Glucagon-like peptide-1 receptor agonist, also called GLP-1 agonist and GLP-1 analog, and includes therapeutics having additional mechanisms of action, such as dual GIP/GLP-1 receptor agonists (for example, tirzepatide).

In some embodiments, the therapeutic having an adverse effect on one or more fascial aesthetics is a SGLT-2 inhibitor. Examples of SGLT-2 inhibitors include canagliflozin (marketed as Invokana), dapagliflozin (marketed as Farxiga), empagliflozin (marketed as Jardiance), bexagliflozin (marketed as Bexacat), sotagliflozin (marketed as Inpefa) and ertugliflozin (marketed as Steglatro).

In some embodiments, the therapeutic having an adverse effect on one or more fascial aesthetics is a corticosteroid. Examples of corticosteroids include prednisone, prednisolone, methylprednisolone, hydrocortisone, hydrocortisone acetate, cortisone acetate, tixocortol pivalate, amcinonide, budesonide, desonide, fluocinolone acetonide, fluocinonide, halcinonide, triamcinolone acetonide, deflazacort (0-isopropylidene derivative), beclometasone, betamethasone, dexamethasone, fluocortolone, halometasone, mometasone, alclometasone dipropionate, betamethasone dipropionate, betamethasone valerate, clobetasol propionate, clobetasone butyrate, fluprednidene acetate, mometasone furoate, ciclesonide, cortisone acetate, hydrocortisone aceponate, hydrocortisone acetate, hydrocortisone buteprate, hydrocortisone butyrate, hydrocortisone valerate, and prednicarbate.

In some embodiments, the therapeutic having an adverse effect on one or more fascial aesthetics is a glucocorticoid. Examples of glucocorticoids include hydrocortisone, cortisone, prednisone, prednisolone, methylprednisolone, meprednisone, triamcinolone, paramethasone, fluprednisolone, betamethasone, and dexamethasone.

In some embodiments, the topical composition stimulates keratinocytes, such as by stimulating activity and/or proliferation of keratinocytes. The topical composition can comprise the agent(s) (e.g., a retinoid such as water-soluble Axerophthol, and/or retinyl palmitate) in an amount effective to stimulate keratinocytes.

In some embodiments, the topical composition increases production of collagen and/or elastin. The topical composition can comprise the agent(s) (e.g., an aliphatic oligopeptide such as Palmitoyl Oligopeptide and/or Palmitoyl Tetrapeptide) in an amount effective to increase production of collagen and/or elastin.

In some embodiments, the topical composition comprises the agent(s) in an amount effective to counteract impairment of keratinocyte inflammatory signaling by the therapeutic.

### Topical Compositions

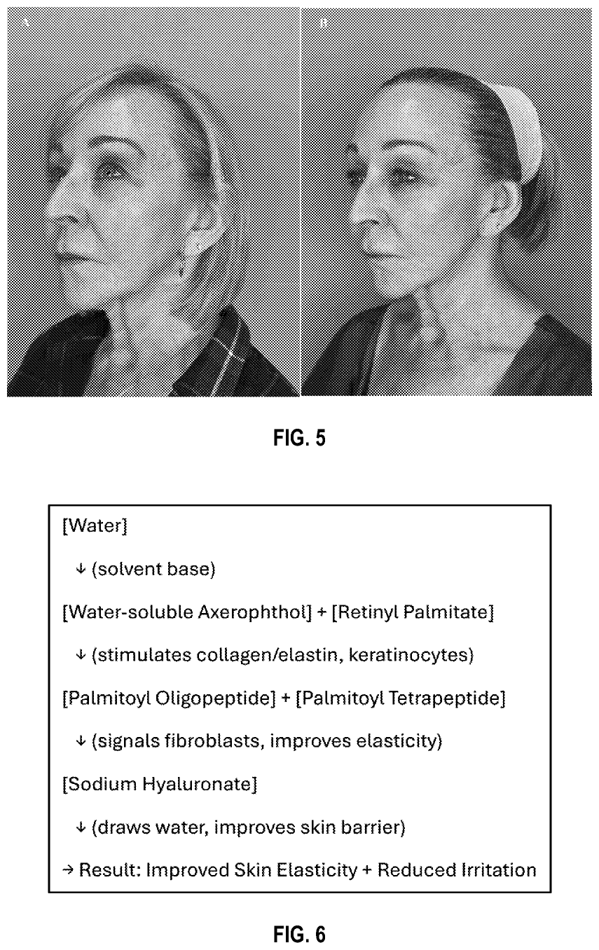

FIG. 6 generally illustrates various components of an embodiment of the present composition and their expected functions. Water is provided as a solvent base. Water-soluble Axerophthol and Retinyl Palmitate stimulates collagen and elastin and keratinocytes. Palmitoyl Oligopeptide and Palmitoyl Tetrapeptide signal fibroblasts and improves elasticity. Sodium Hyaluronate draws water and improves skin barrier. The result is improved skin elasticity and reduced irritation.

In some embodiments, topical composition is a cosmetic composition or a cosmeceutic composition. The term “cosmetic” refers to a material or action intended to restore or improve a person's appearance, such as preventing or reducing the signs of aging skin. Signs of aging skin include, in some instances, wrinkles, fine lines, blemishes, skin discoloration, dry skin, skin irritation, saggy skin, inelastic skin, enlarged pores, acne scars, inflammation, crow's feet, laugh lines, drooping eyelids, crepey skin, frown lines, dull skin tone, dark circles under the eyes, lackluster skin, itchy skin, hyperpigmentation, uneven skin tone, and collagen loss. The term “cosmeceutic” refers to a material or action with bioactive ingredients that claim to enhance skincare efficacy, offering a bridge between over-the-counter cosmetics and prescription products.

The topical compositions comprise a retinoid in an aqueous medium. The compositions can also comprise one or more (or all) of an aliphatic oligopeptide; an acetylated oligopeptide; and hyaluronic acid. The present compositions include one or more retinoids. Retinoids have been used for treating aging, burns, scaling, and dermatitis and are believed to regulate epithelial cell growth. Examples of retinoids include retinol, retinal (also called retinaldehyde), retinyl acetate, retinyl esters of fatty acids (such as retinyl palmitate, retinyl oleate, retinyl stearate, and retinyl linoleate), tretinoin, alitretinoin, isotretinoin, bexarotene, adapalene, and tazarotene. In some embodiments, the retinoid is selected from the group consisting of retinol (also called axerophthol and vitamin A1), retinal (also called vitamin A-aldehyde), retinyl esters (also called vitamin A-ester), and mixtures thereof. Retinol is sometimes referred to as Vitamin A, Vitamin A alcohol, Vitamin A1, or all-trans-3,7-Dimethyl-9-(2,6,6-trimethyl-1-cyclohexen-1-yl)-2,4,6,8-nonatetraen-1-ol.

Retinoids such as Vitamin A and its derivatives are among the most effective substances slowing the aging process. Through activation of retinoid nuclear receptors, retinoids regulate cellular apoptosis, differentiation and proliferation. Anti-wrinkle properties of retinoids promote keratinocytes proliferation, strengthen the protective function of the epidermis, restrain transepidermal water loss, protect collagen against degradation, promote collagen biosynthesis, and inhibit metalloproteinases activity (Zasada 2019; Hubbard 2014). Increased collagen content may then indirectly stimulate normalization of the elastic tissue organization (Hubbard 2014).

Retinol is fat soluble and can penetrate the stratum corneum and slightly into the dermis. Retinol is effective for stimulating keratinocytes which are cells found in the outermost layer of the skin and form a barrier against damage. Keratinocytes differentiate from epidermal stem cells in the lower part of the epidermis (stratum basale) and migrate towards the surface. When retinol enters into a keratinocyte, it binds CRBP or is transformed into more active metabolites, such as retinoic acid, which binds to CRABP2, enters into the nucleus, and activates its retinoid nuclear receptor. (Zasada 2019; Bouriez) The retinoid nuclear receptors, RAR and RXR, form heterodimers and function as transcriptional regulators. Activated RAR/RXR heterodimers can also interact with other transcription factors, such as AP-1, estrogen receptor a, and NF-kB (Wang 2007).

At low concentrations, retinoic acid prevents binding of NRF2 to the ARE, thereby suppressing the expression of NRF2-target antioxidative genes (Wang 2007). Furthermore, RXR can inhibit the transcriptional activity of NRF2 through associating with ARE-bound NRF2, suggesting that inhibition of NRF2 by RXR arises from direct interference of recruitment of coactivators to gene promoters (Wang 2013).

The present compositions can comprise one or more retinoids at a concentration (on a weight basis) of at least 0.01%, or at least 0.1%, or at least 0.5%, or at least 0.75%, or at least 1%, or at least 1.1%, or at least 1.5%, or at least 2%, or at least 3%, or at least 5%, or at least 10%. The present compositions can comprise one or more retinoids at a concentration (on a weight basis) of at most 20%, or at most 15%, or at most 10%, or at most 5%, or at most 4%, or at most 3%, or at most 2.5%, or at most 2%. Any of the foregoing minimum and maximum may be combined to form a desired concentration range, so long as the minimum is smaller than the maximum.

The present compositions include one or more aliphatic oligopeptides, such as palmitoyl tetrapeptide-7 ((2S)-2-[[(2S)-1-[(2S)-5-amino-2-[[2-(hexadecanoylamino)acetyl]amino]-5-oxopentanoyl]pyrrolidine-2-carbonyl]amino]-5-(diaminomethylideneamino)pentanoic acid, also referred to as Pal-Gly-Gln-Pro-Arg-OH or CAS No. 221227-05-0) or palmitoyl tripeptide-1 (N-(1-oxohexadecyl)glycyl-L-histidyl-L-lysine, also referred to as Pal-Gly-His-Lys-OH or CAS No. 147732-56-7). Aliphatic oligopeptides improve penetration of the peptide-containing molecule through lipid structures of the skin and are believed to activate certain genes involved in the process of extracellular matrix renewal and cell proliferation. By activating the neosynthesis of extracellular matrix macromolecules, palmitoyl pentapeptide-4 may provide an anti-wrinkle effect. Various embodiments of the present compositions can include a selected amount of one or more aliphatic oligopeptides, such as at least about 0.5%, 1%, 2%, or 3% by weight, or at most about 15%, 10%, 7% or 5%; any of these minimums and maximums can be combined to form a range for the amount of aliphatic oligopeptides.

The present compositions include hyaluronic acid. Hyaluronic acid has been used as a humectant, as it promotes hydration and can hold many times its weight in water. The terms “hyaluronic acid” and “hyaluronate” as used herein are generally equivalent, unless the context indicates otherwise, such as when a specific hyaluronate salt is specified. Furthermore, the terms “hyaluronic acid” and “hyaluronate” generally include hyaluronan, hyaluronate, and glycosaminoglycan. In some embodiments, the present compositions contain a high molecular weight hyaluronic acid and/or a hyaluronic acid which is released over time. In some embodiments, the present compositions contain a selected amount of hyaluronic acid, such as from about 0% to about 5% by weight hyaluronic acid. In some embodiments, the compositions contain at most about 0.1%, or 0.25%, or 0.5%, or 1%, or 1.25%, or 1.5%, or 1.75%, or 2%, or 2.5%, or 3.0% hyaluronic acid. In some circumstances, the foregoing percentages of hyaluronic acid may be on a volume basis.

In some embodiments, the present compositions include one or more humectants (in addition to hyaluronic acid). Humectants can be added to cause increased elasticity, smoothness, and hydration of the skin. Examples of additional humectants include alkyl diols and polyols, alkenyl diols and polyols, alkoxy glycosides, glyceryl triacetate and glycerol polymers, sugars (sucrose, trehalose, fructose, glucose, maltose, etc.), sugar alcohols (erythritol, glycerol, sorbitol, xylitol, maltitol, mannitol, inositol, lactitol, etc.), alkoxy derivatives of sugars and sugar alcohols, alpha hydroxy acids such as lactic acid, and ureas. In some embodiments, the additional humectant is selected from the group consisting of glycerin; propane diols (such as methyl propane diol); propylene glycols (such as dipropylene glycol, triethylene glycol); short-chain polyethylene glycols (<200 mol wt); butylene glycols; pentylene glycols; hexylene glycols (such 1,2,6-hexanetriol); sorbitol, urea compounds; sodium pyrrolidone carboxylic acid; chitosan; and C6-10 alkane diols (such as 1,2-hexanediol, 1,2-octanediol (also called caprylyl glycol), 1,9-nonanediol, 1,2-decanediol, and 1,10-decanediol), and mixtures thereof. In some embodiments, the present compositions contain a selected amount of additional humectant, such as from about 0.05% to about 5% by volume. In some embodiments, the compositions contain at least 0.1% by volume, or 0.25%, or 0.5%, or at most about 2% by volume, or 1%; any of these minimums and maximums can be combined to form a range for the amount of humectant.

In some embodiments, the present compositions can also include any one of, any combination of, or all of the following additional ingredients: a chelating agent, a UV absorption agent, a moisturizing agent, a preservative, a thickening agent, a silicone containing compound, a structuring agent, a vitamin, a pharmaceutical ingredient, or an antioxidant, or any combination of such ingredients or mixtures of such ingredients. In some embodiments, the composition can include at least two, three, four, five, six, seven, eight, nine, ten, or all of these additional ingredients.

In some embodiments, the compositions comprise a solvent in a selected amount. Examples of solvents include water, glycerin, propylene glycol, propanediol, butylene glycol, ethoxydiglycol, isododecane, isohexadecane, octyldodecanol, hexylene glycol, 1,2-hexane diol, and dicaprylyl carbonate, and mixtures thereof. In some embodiments, the solvent is water. The compositions can contain at least 50%, or 60%, or 65%, or 70%, or 75%, or 80% by weight. In some embodiments, the present compositions is about 65 to 70% water. In some circumstances, the foregoing percentages of water may be on a volume basis. In some embodiments, the present compositions are an aqueous composition having a single phase or a homogeneous composition. In some embodiments, the present compositions are an emulsion, such as an oil-in-water emulsion or a water-in-oil emulsion. In some embodiments, the emulsion is a multiple emulsion, such as water-in-oil-in-water emulsion or an oil-in-water-in-oil emulsion.

In some embodiments, the present compositions comprise a fatty alcohol emollient. Examples of fatty alcohol emollients include capryl alcohol, pelargonic alcohol, capric alcohol, lauryl alcohol, myristyl alcohol, cetyl alcohol, isocetyl alcohol, stearyl alcohol, isostearyl alcohol, cetearyl alcohol, oleyl alcohol, ricinoleyl alcohol, arachidyl alcohol, icocenyl alcohol, behenyl alcohol, and mixtures thereof such as cetearyl alcohol. In some embodiments, the present compositions contain a selected amount of a fatty alcohol emollient, such as from about 0.5% to about 15% by weight. In some embodiments, the compositions contain at least 1% by weight, or 2%, or 3%, or at most about 10% by weight, or 7% w/w, or 5% w/w; any of these minimums and maximums can be combined to form a range for the amount of fatty alcohol emollient. In some circumstances, the foregoing percentages of fatty alcohol emollient may be on a volume basis.

In some embodiments, the present compositions comprise an emulsifier. Emulsifiers can reduce the interfacial tension between phases and improve the formulation and stability of an emulsion. The emulsifiers can be nonionic, cationic, anionic, and zwitterionic emulsifiers. Non-limiting examples include esters of glycerin, fatty acid esters of glycerol such as glyceryl stearate, esters of propylene glycol, fatty acid esters of polyethylene glycol and/or polypropylene glycol such as PEG-100 stearate, salts of fatty acids such as TEA stearate, esters of sorbitol, esters of sorbitan anhydrides, carboxylic acid copolymers, esters of glucose, ethers of glucose, ethoxylated ethers, ethoxylated alcohols, alkyl phosphates, polyoxyethylene fatty ether phosphates, fatty acid amides, acyl lactylates, DEA oleth-3 phosphate, polyethylene glycol 20 sorbitan monolaurate (polysorbate 20), polyethylene glycol 5 soya sterol, steareth-2, steareth-20, steareth-21, ceteareth-20, cetearyl glucoside, cetearyl alcohol, C12-13 pareth-3, PPG-2 methyl glucose ether distearate, PPG-5-ceteth-20, bis-PEG/PPG-20/20 dimethicone, ceteth-10, polysorbate 80, cetyl phosphate, potassium cetyl phosphate, diethanolamine cetyl phosphate, polysorbate 60, arachidyl alcohol, arachidyl glucoside, and mixtures thereof. In some embodiments, the present compositions contain a selected amount of an emulsifier, such as from about 0.5% to about 20% by weight. In some embodiments, the compositions contain at least 1% by weight, or 2%, or 3% of emulsifier, or at most about 15% by weight, or 10%, or 5% of emulsifier; any of these minimums and maximums can be combined to form a range for the amount of emulsifier. In some circumstances, the foregoing percentages of emulsifier may be on a volume basis.

In some embodiments, the present compositions comprise a silicone oil. Examples of silicone oils such as a polyorganosiloxane. Non-limiting examples of polyorganosiloxanes include dimethicone, cyclomethicone, polysilicone-11, phenyl trimethicone, trimethylsilylamodimethicone, stearoxytrimethylsilane, or mixtures thereof. In some embodiments, the present compositions contain a selected amount of a silicone oil, such as from about 0.5% to about 10% by weight. In some embodiments, the compositions contain at least 1% by weight, or 2%, or 3%, or at most about 7% by weight, or 5%; any of these minimums and maximums can be combined to form a range for the amount of silicone oil. In some circumstances, the foregoing percentages of silicone oil may be on a volume basis.

In some embodiments, the present compositions comprise a fragrance, such as a natural oil or a synthetic fragrance. Examples of fragrances include apricot (Prunus armeniaca), chamomile (Anthemis nobilis), coconut (Cocos nucifera), grape (Vitis vinifera), jasmine (Jasminum officinale), lavender (Lavandula angustifolia), lemon (Citrus medica limonum), orange (citrus Aurantium dulcis), peach (Prunus persica), peppermint (Mentha piperita), sage (Salvia officinalis), sandalwood (Santalum album), and combinations thereof. In some embodiments, the present compositions contain a selected amount of a fragrance, such as from about 0.05% to about 5% by weight. In some embodiments, the compositions contain at least 0.1% by weight, or 0.25%, or 0.5%, of fragrance, or at most about 2% by weight, or 1%, of fragrance; any of these minimums and maximums can be combined to form a range for the amount of fragrance.

In some embodiments, the present compositions comprise a preservative. Examples of preservatives include benzalkonium halides such benzalkonium chloride, parabens such as methylparabens and propylparabens, phenoxyethanol, benzyl alcohol, chlorobutanol, phenol, sorbic acid and salts thereof such potassium sorbate, or combinations of two, three, or more of those preservatives. In some embodiments, the present compositions contain a selected amount of a preservative, such as from about 0.05% to about 5% by weight. In some embodiments, the compositions contain at least 0.1% by weight, or 0.25%, or 0.5% of a preservative, or at most about 2% by weight, or 1% of a preservative; any of these minimums and maximums can be combined to form a range for the amount of preservative.

The present compositions can include one or more physiologically acceptable excipients. The term “physiologically acceptable” refers to compounds and materials which are suitable for use in contact with the tissues of human beings and/or other mammals without excessive toxicity, irritation, allergic response, or other problem or complication, commensurate with a reasonable benefit/risk ratio. In some aspects, physiologically acceptable means approved by a regulatory agency of the federal or a state government, or listed in the U.S. Pharmacopeia or other generally recognized pharmacopeia for use in mammals, and more particularly, in humans.

The present compositions can also include other physiologically acceptable excipients which may fall into one or more types. For example, some excipients may be considered both emollients and emulsifiers. Persons skilled in the field of cosmetic formulations will recognize that the identification of an excipient as being within one type does not exclude that same excipient as being within another type. In some embodiments, an excipient may be one type when combined with certain excipients, and another type when used in combination with other excipients.

The present topical compositions can be formulated as serums, creams, gels, lotions, or ointments. Serums are generally light liquids easily absorbed by the skin. The present serum is designed to be readily absorbed by the skin surface and produce a highly bioavailable effect on the tissue. Creams are generally semi-solid emulsions of oil and water in approximately equal proportions, and can be oil-in-water (O/W) or water-in-oil (W/O) creams. Gels are semi-solid dispersions of liquid or oil particles in a semi-solid medium. Lotions are generally lower in viscosity than creams and gels, and are often oil-in-water emulsions containing an emulsifier such as cetyl alcohol to prevent phase separation. Ointments are generally compositions having a relatively high oil content, such as an oil:water ratio of from 7:1 to 2:1.

The present compositions can have a desired viscosity which reflects its formulation or its intended use. Depending on the formulation or intended use, the viscosity of the composition can be from about 1 cps to about 1 million cps, or any range based on combining any of the following values: 2 cps, 3, 4, 5, 6, 7, 8, 9, 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, 10000, 20000, 30000, 40000, 50000, 60000, 70000, 80000, 90000, 100000, 200000, 300000, 400000, 500000, 600000, 700000, 800000, 900000, 1000000, 2000000, 3000000, 4000000, 5000000, 10000000, cps. The foregoing viscosities can be determined on a Brookfield Viscometer using a TC spindle at 2.5 rpm at 25 degrees C.

### Dermatological Procedures

As yet another aspect of the present invention, methods are provided for improving one or more facial aesthetics of a subject. The methods comprise applying a topical composition to facial skin of a subject identified as receiving or about to receive a therapeutic having an adverse effect on one or more facial aesthetics. The methods also comprise treating the facial skin of the subject by a dermatological procedure before, during or after applying the topical composition. In some embodiments, the dermatological procedure is selected from the group consisting of microdermabrasion, hydradermabrasion, dermabrasion, and dermal planing. For example, the dermatological procedure can include abrading a surface of facial skin of a subject to remove dead cells and/or cell debris from the skin surface, and this abrading of the facial skin is performed before or during the application of the topical composition to the facial skin. The abrading can be performed by techniques such as microdermabrasion and hydradermabrasion. In some embodiments, the topical composition is applied to the facial skin by infusion, or by injection or microinjection.

In some embodiments, the present compositions are applied to a subject's facial skin before, during or after a dermatological procedure. Accordingly, the present technology includes methods of improving one or more facial aesthetics of a subject by applying the present composition to the subject's facial skin, and treating the subject's facial skin by one or more other techniques, such as microdermabrasion, hydradermabrasion, dermabrasion, dermal planing, massage therapy, administering botulin or other neurotoxins, cosmetic surgery such as brow lifts and blepharoplasty, fillers, micro-needling, peels, exfoliations, suctioning, fluid delivery, acid treatments, extractions, energy-based and other treatments, such as lasers, thermal, radiofrequency, light (e.g., photofacials/IPL), such as light therapy, photodynamic therapy, radio frequency (RF) therapy, or microwave energy therapy. For example, the dermatological procedure can include abrading a surface of facial skin of a subject to remove dead cells and/or cell debris from the skin surface, and this abrading of the facial skin is performed before or during the application of the topical composition to the facial skin. The abrading can be performed by techniques such as microdermabrasion and hydradermabrasion. In some embodiments, the topical composition is applied to the facial skin by infusion, or by injection or microinjection.

Abrasion as used herein refers to removal or loss of skin tissue and is generally superficial (involving only the epidermis or a sublayer such as the stratum corneum) but in some instances may be deep (involving the deep dermis). Abrasion of the outer layer of the skin or epidermis has been used to smooth or blend scars, blemishes, or other skin conditions. Techniques used for abrasion include dermabrasion and microdermabrasion, which remove portions of the epidermis called the stratum corneum. The body then replaces the lost skin cells, resulting in a new outer layer of skin. Dermabrasion devices may include a wire brush, wheel, sterilized sandpaper, crystals, or other mechanical means.

Microdermabrasion is a technique to temporarily remove top layers of the skin called the stratum corneum, thereby removing skin debris and/or dead cells from the epidermis.

Microdermabrasion uses an exfoliating medium which is applied pneumatically to remove the outermost layer of skin debris and dead cells from the epidermis. Most commonly, microdermabrasion uses two parts: an exfoliating medium comprising crystals or particles, and a pneumatic device applying a vacuum or suction to gently lift up the skin during exfoliation.

Since there may be minor skin sensitivity after microdermabrasion, irritating products like glycolic acids, alpha hydroxy acids, retinoid products, or fragranced creams and lotions are typically avoided right after the procedure. The American Society for Dermatologic Surgery states that glycolic, beta hydroxy acids, retinol, benzyl peroxide and similar medications should be avoided for one to two days following treatment, and applying prescription retinoid products should be avoided for three to seven days after treatment.

Hydradermabrasion combines microdermabrasion with pneumatic application of a composition such as a serum. The serum typically includes an antioxidant. Antioxidants introduced through the procedure hydrate and decrease inflammation in the skin, reversing photo damage, while protecting lipid membranes, collagen fibers, and enzyme systems.

In some embodiments, the present compositions can be used in a dermal infusion treatment. Dermal infusion delivers a composition under pressure to treat dermatological conditions. It has been used to treat rosacea, dehydration, acne, and post-inflammatory hyperpigmentation. In some embodiments, the present composition, in the form of an aqueous suspension or serum, can be filled into a known dermal infusion device and then applied to the skin. The present composition may be applied with or without microdermal abrasion or skin suction.

In some embodiments, the present methods comprise at least partial abrasion of facial skin before, during or after applying the present composition. For instance, a delivery device may comprise one or more abrading members (e.g., posts, spiral, ridges, brushes, sharp edges, roughened surfaces, etc.). When the device is activated (e.g., during the delivery of pulsed air and/or during the maintenance of suction through corresponding vacuum ports, etc.), the user can selectively translate or move the tip of the device relative to the targeted skin surface to at least partially abrade and/or otherwise treat the skin. In other embodiments, the dermal infusion device comprises a non-abrading member for the purpose of driving serums and/or other fluids deeper into the skin. For example, such a non-abrading member can be used following an abrading or preparatory procedure, step or phase.

In some embodiments, the present compositions can be applied using a delivery device, such as the devices described in Ignon et al. US Pat. App. Publication No. 2007/0156124 A1; Boone et al. US Pat. App. Publication No. 2010/0049177 A1; Chang et al. US Pat. App. Publication No. 2015/0088050 A1; Richardson US Pat. App. Publication No. 2018/0140317 A1; Shadduck et al. US Pat. App. Publication No. 2018/0303515 A1; Ignon et al. US Pat. App. Publication No. 2019/0133642 A1; and Straka US Pat. App. Publication No. 2019/0191937 A1.

Before the various examples are described, it is to be understood that the teachings of this disclosure are not limited to the particular examples described, and as such can, of course, vary. In view of this disclosure it is noted that the compositions and methods can be implemented in keeping with the present teachings. Further, various components, materials, steps and parameters are included by way of illustration and example only and not in any limiting sense. In view of this disclosure, the present teachings can be implemented in other applications and components, materials, structures and equipment to implement these applications can be determined, while remaining within the scope of the appended claims.

### EXAMPLES

### Example 1

In this example, an embodiment of the present composition is formulated. The composition, GLPSGLT 1 serum (Afore LLC, Chicago, IL), was designed to provide bioavailable local form of retinoic acid to the dermis, theoretically helping to partially reverse cellular and cytokine disruption [14]. The GLPSGLT serum contains a proprietary blend of water, retinoic acid, peptides, sodium hyaluronate, and botanically derived ingredients.

TABLE 1

| Name | CAS No. | % |
|---|---|---|
| Purified Water | 1314-13-2 | 70-80 |
| Sodium Hyaluronate | 9067-32-7 | 3-5 |
| Aloe Vera Gel | 85507-69-3 | 3-5 |
| Chamomile | 8015-92-7 | 1-3 |
| Calendula | 84776-23-8 | 1-3 |
| Ascorbic Acid | 50-81-7 | 1-3 |
| Lecithin | 8002-43-5 | 1-3 |
| Palmitoyl Oligopeptide | 147732-56-7 | 1-3 |
| Palmitoyl Tetrapeptide | 221227-05-0 | 1-3 |
| Sodium PCA | 28874-51-3 | 0.1-1 |
| Carbomer | 9007-20-9 | 0.1-1 |
| Hydroexyethyl Cellulose | 9004-62-0 | 0.1-1 |
| Ascorbyl Palmitate | 137-66-6 | 0.1-1 |
| Ginseng Extract | 50647-08-0 | 0.1-1 |
| Allantoin | 97-59-6 | 0.1-1 |
| Guar Gum | 9000-30-0 | 0.1-1 |
| Retinol | 68-26-8 | 0.1-1 |
| Retinyl Palmitate | 79-81-2 | 0.1-1 |
| Phenoxyethanol | 122-99-6 | 0.1-1 |
| Potassium Sorbate | 24634-61-5 | 0.1-1 |
| Citric Acid | 77-92-9 | 0.1-1 |

### Example 2

In this example, the cosmetic efficacy, safety and tolerability of the composition of Example 1 when administered to facial skin was studied. The composition was formulated as a topical serum. This study represents the first placebo controlled study to evaluate a topical serum use to enhance the elasticity, barrier function, and textural changes seen in the GLP-1 RA patient.

### Study Design

The study was a single-center, split face, blinded, pilot study to assess the clinical effects of a GLPSGLT topical serum when applied twice daily to facial skin. This study was approved by Advarra institution review board prior to initiation.

A total of 7 patients were enrolled, with the characteristic shown in Table 2.

TABLE 2
Subject Characteristics (N = 7)

| Characteristic | Value | |
|---|---|---|
| Median Age, years (range) | 55, (34-64) | |
| GRS Skin Type, n (%) | | |
| I: pale white or freckled | 5 | (71.4) |
| II: white | 0 | (0.0) |
| III: white to light brown | 0 | (0.0) |
| IV: moderate brown | 1 | (14.3) |
| V: dark brown | 1 | (14.3) |
| VI: very dark brown to black | 0 | (0.0) |
| GLP-1 or SGLT Drug, n (%) | | |
| tirzepatide | 3 | (42.8) |
| semaglutide | 3 | (42.8) |
| dulaglutide | 1 | (14.3) |

Patients were selected who met inclusion criteria based on skin quality related to GLP-1 analog therapy, SGLT-2 therapy, and aging. The inclusion criteria were healthy females or males of ages between 18 and 75 years old receiving GLP-1 analog or SGLT-2 therapy for a minimum of 30 days prior to the start of the study. The inclusion criteria for skin quality were presenting with at least one of the following: volume loss, loss of elasticity, presence of fine lines and wrinkles, enlarged pores, photodamage, surface roughness, and uneven pigmentation. Patients who had received botulinum toxin A, filler, or energy-based treatments within 3 months of study enrollment or who had undergone rejuvenation procedures to the face or neck (e.g. facelift surgery, microneedling, microdermabrasion, chemical peels) within 30 days of the start of the study were excluded. Those who were current smokers, had a history of heavy smoking, had uncontrolled systemic inflammatory conditions, or had active dermatologic conditions in the areas to be treated were also excluded. For the duration of the study, patients did not receive any aesthetic treatments on their face or neck, and their use of topical products was limited to the product under study, the placebo topical, a gentle facial cleanser, a daily facial moisturizer, and routine 25% zinc-oxide based SPF 30 sunscreen application daily as recommended by the American Academy of Dermatology.

In this pilot study, the patients served as their own placebo control group using a split-face study design. Following baseline assessment, each patient was provided with two bottles of product in unbranded amber glass containers with pumps: one labeled “LEFT” by the product manufacturer, and one labeled “RIGHT” by the product manufacturer. The LEFT product contained the components of Table 1, and the RIGHT product contained water, Aloe Vera gel, sodium hyaluronate, phenoxyethanol, and potassium sorbate.

Subjects were instructed to apply a thin layer of the serum labeled “LEFT” to the left side of their face following use of the provided mild facial cleanser on the entire face in both the morning and evening. After applying the topical labeled “LEFT”, subjects were instructed to wash their hands, then apply a thin layer of the topical product labeled “RIGHT” to the right side of their face. In the mornings, subjects were instructed to apply a thin layer of the daily facial moisturizer to both sides of the face, followed by a thin layer of the 25% zinc-oxide based sunscreen, after applying both “LEFT” and “RIGHT” topicals. This regimen was continued for 42 days (6 weeks) with follow-up visits at days 21 and 42.

Compliance was ensured by weighing the product containers at the end of the study. Patients also filled out a diary in which they recorded each application of product, as well as recorded any changes to their skin appearance and perceived side effects. Patients were not reimbursed for their time, but the cost of parking at the study facility was covered.

### Efficacy and Safety Measures

### Primary Endpoint

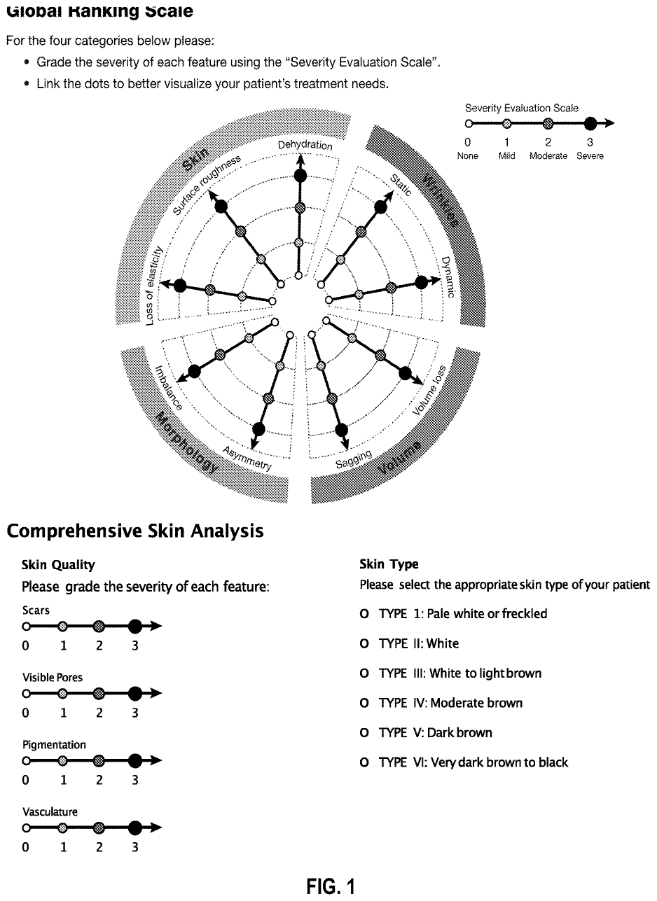

The evaluators, clinical staff, and subjects were blinded to placebo versus active study product. An off site clinical coordinator, not involved in the assessment, was the control monitor. At baseline and each follow-up visit, standardized photographs of the treatment areas were captured. Patients were assessed live (co-assessment included both the patient and the treating clinician) using the Global Ranking Scale (GRS) which is illustrated in FIG. 1 with Comprehensive Skin Analysis [15]. The primary endpoint of this pilot study was the difference in the GRS between sides of the face compared to baseline. The GRS assessments were completed for the entire face on day 1 (baseline). In addition, the GRS was conducted separately for each side of the face on days 21, and 42. Both subjects and the investigator were blinded to the treatment vs. placebo sides of the face. For each of the 13 domains included in the GRS (loss of elasticity, surface roughness, dehydration, static wrinkles, dynamic wrinkles, volume loss, sagging, asymmetry, imbalance, scar presence, visible pores, pigmentation, and vasculature) grading ranges from 0 to 3 (0, none; 1, mild; 2, moderate; 3, severe). The patient and investigator were blinded to baseline scores and to any prior follow up scores at each evaluation. The protocol was based on a validated protocol from previous published study in the Aesthetic Surgery Journal [16].

### Independent Photographic Review

In addition to the GRS, an independent review of standardized before-and-after images was carried out by a blinded board-certified plastic surgeon working in a remote clinic to the study site. The independent reviewer, who was blinded to the patient GRS scores for both the treatment and placebo sides of the face, was provided with patient images at day 1 (baseline) and 42 days, and asked to select which side (left or right) showed significant improvement between days 1 and 42, or select “no difference” if no qualitative difference was observed. The evaluator was asked to only review and select a side for domains that could be assessed in a remote setting; static wrinkles, scars, vasculature, surface roughness, and pigmentation. The remaining GRS domains were excluded: dynamic wrinkles, loss of elasticity, sagging, volume loss, asymmetry, and dehydration. The percentage of patients considered improved in each domain was then calculated.

### Secondary Endpoint

A 26-question Subject Satisfaction Questionnaire was completed independently by patients, at home, prior to their office visits on days 21 and 42. The questionnaire asked the subjects twenty-six questions, twenty-four of which were closed questions with assigned numerical values, and two of which were open-ended questions. The content of the Questionnaire at 3 weeks (21 days) is shown below; the content of the Questionnaire at 6 weeks (42 days) was the same, except for the weeks/days of the questionnaire.

| | |
|---|---|
| 1. Overall, I am very satisfied with the | 2. Overall, I am very satisfied with the |
| results from the topical labeled | results from the topical labeled |
| “LEFT” after 3 weeks (21 days) | “RIGHT” after 3 weeks (21 days) |
| Strongly Agree (5) | Strongly Agree (5) |
| Agree (4) | Agree (4) |
| Neutral (3) | Neutral (3) |
| Disagree (2) | Disagree (2) |
| Strongly Disagree (1) | Strongly Disagree (1) |
| 3. I felt that there was a visible reduction in | 4. I felt that there was a visible reduction in |
| the fine lines and wrinkles on the LEFT | the fine lines and wrinkles on the RIGHT |
| side of my face after 3 weeks (21 days) | side of my face after 3 weeks (21 days) |
| Strongly Agree (5) | Strongly Agree (5) |
| Agree (4) | Agree (4) |
| Neutral (3) | Neutral (3) |
| Disagree (2) | Disagree (2) |
| Strongly Disagree (1) | Strongly Disagree (1) |
| 5. The study topical labeled “LEFT” | 6. The study topical labeled “RIGHT” |
| made me feel more confident in the | made me feel more confident in the |
| physical appearance of my face after | physical appearance of my face after |
| 3 weeks (21 days) | 3 weeks (21 days) |
| Strongly Agree (5) | Strongly Agree (5) |
| Agree (4) | Agree (4) |
| Neutral (3) | Neutral (3) |
| Disagree (2) | Disagree (2) |
| Strongly Disagree (1) | Strongly Disagree (1) |
| 7. I felt there was a significant | 8. I felt there was a significant |
| improvement in the texture/smoothness | improvement in the texture/smoothness |
| of the skin treated with the serum | of the skin treated with the serum |
| labeled “LEFT” after 3 weeks (21 days) | labeled “RIGHT” after 3 weeks (21 days) |
| Strongly Agree (5) | Strongly Agree (5) |
| Agree (4) | Agree (4) |
| Neutral (3) | Neutral (3) |
| Disagree (2) | Disagree (2) |
| Strongly Disagree (1) | Strongly Disagree (1) |
| 9. I felt there was a significant | 10. I felt there was a significant |
| improvement in my pore size on the skin | improvement in my pore size on the skin |
| treated with the serum labeled “LEFT” | treated with the serum labeled “RIGHT” |
| after 3 weeks (21 days) | after 3 weeks (21 days) |
| Strongly Agree (5) | Strongly Agree (5) |
| Agree (4) | Agree (4) |
| Neutral (3) | Neutral (3) |
| Disagree (2) | Disagree (2) |
| Strongly Disagree (1) | Strongly Disagree (1) |
| 11. I felt there was a significant | 12. I felt there was a significant |
| improvement in the firmness of my skin | improvement in the firmness of my skin |
| treated with the serum labeled “LEFT” | treated with the serum labeled “RIGHT” |
| after 3 weeks (21 days) | after 3 weeks (21 days) |
| Strongly Agree (5) | Strongly Agree (5) |
| Agree (4) | Agree (4) |
| Neutral (3) | Neutral (3) |
| Disagree (2) | Disagree (2) |
| Strongly Disagree (1) | Strongly Disagree (1) |
| 13. I felt there was a significant | 14. I felt there was a significant |
| improvement in the radiance/luminosity | improvement in the radiance/luminosity |
| of my skin after using the serum labeled | of my skin after using the serum labeled |
| “LEFT” for 3 weeks (21 days) | “RIGHT” for 3 weeks (21 days) |
| Strongly Agree (5) | Strongly Agree (5) |
| Agree (4) | Agree (4) |
| Neutral (3) | Neutral (3) |
| Disagree (2) | Disagree (2) |
| Strongly Disagree (1) | Strongly Disagree (1) |
| 15. I felt there was a significant | 16. I felt there was a significant |
| improvement in my skin tone and | improvement in my skin tone and |
| evenness after using the serum labeled | evenness after using the serum labeled |
| “LEFT” for 3 weeks (21 days) | “RIGHT” for 3 weeks (21 days) |
| Strongly Agree (5) | Strongly Agree (5) |
| Agree (4) | Agree (4) |
| Neutral (3) | Neutral (3) |
| Disagree (2) | Disagree (2) |
| Strongly Disagree (1) | Strongly Disagree (1) |
| 17. I felt there was a significant | 18. I felt there was a significant |
| improvement in my skin moisture after | improvement in my skin moisture after |
| using the serum labeled “LEFT” for | using the serum labeled “RIGHT” for |
| 3 weeks (21 days) | 3 weeks (21 days) |
| Strongly Agree (5) | Strongly Agree (5) |
| Agree (4) | Agree (4) |
| Neutral (3) | Neutral (3) |
| Disagree (2) | Disagree (2) |
| Strongly Disagree (1) | Strongly Disagree (1) |
| 19. The serum labeled “LEFT” felt | 20. The serum labeled “RIGHT” felt |
| comfortable and soothing on my skin | comfortable and soothing on my skin |
| Strongly Agree (5) | Strongly Agree (5) |
| Agree (4) | Agree (4) |
| Neutral (3) | Neutral (3) |
| Disagree (2) | Disagree (2) |
| Strongly Disagree (1) | Strongly Disagree (1) |
| 21. The serum labeled “LEFT” is gentle | 22. The serum labeled “RIGHT” is gentle |
| enough for everyday use | enough for everyday use |
| Strongly Agree (5) | Strongly Agree (5) |
| Agree (4) | Agree (4) |
| Neutral (3) | Neutral (3) |
| Disagree (2) | Disagree (2) |
| Strongly Disagree (1) | Strongly Disagree (1) |
| 23. I would recommend the serum labeled | 24. I would recommend the serum labeled |
| “LEFT” to family and friends | “RIGHT” to family and friends |
| Strongly Agree (5) | Strongly Agree (5) |
| Agree (4) | Agree (4) |
| Neutral (3) | Neutral (3) |
| Disagree (2) | Disagree (2) |
| Strongly Disagree (1) | Strongly Disagree (1) |
| 25. Have you noticed any other aesthetic | 26. Have you noticed any other aesthetic |
| effects of the serum labeled “LEFT” | effects of the serum labeled “RIGHT” |
| over the past 3 weeks (21 days) of | over the past 3 weeks (21 days) of |
| applying the product? | applying the product? |
| Other Comments | Other Comments |

### Safety

Safety was monitored via a virtual visit on day 3 and at each follow-up visit (days 21 and 42) through assessment of the facial skin by the investigator and by review of patient diaries. This study was approved by an Institutional Review Board (Advarra IRB, Columbia, MD) and adhered to the standards set forth in the World Medical Association's Declaration of Helsinki. Written consent was provided, by which the patients agreed to the use and analysis of their data and publication of their photographs.

### Results

The study enrolled 7 patients (all female) with a median age of 55 years (range 34-64 years) (Table 2). Participants were using various GLP-1 RA medications, including 3 subjects [42.8%] using tirzepatide (Zepbound, Mounjaro), 3 subjects [42.8%] using semaglutide (Ozempic, Wegovy), and 1 subject [14.3%] using dulaglutide (Trulicity). Patients were fully compliant with completion of all GRS evaluations and patient questionnaires. Patients with a wide range of Fitzpatrick skin types were included in this study (Table 2). All subjects maintained a stable weight within 5 pounds.

To assess the GRS, the difference from baseline was calculated for each subject at each time point for each side (ABL Right and ABL Left). The calculated results are shown in Table 3, with mean differences and p-values for each feature. Matched pairs was used to test if the difference between sizes was equal to zero. In all cases, the difference between the treated and untreated sides was greater than zero, indicating that the GRS scores were better for the treated side. These differences were statistically significant with both a t-test and a signed rank test. Note, no correction was made for multiple tests, however using a simple Bonferroni correction of 0.05/13=0.0038 does not change the conclusion that the GRS scores are better for the treated side as compared to the untreated side.

TABLE 3

| | ΔBL Left − | | |
|---|---|---|---|
| | ΔBL Right | t Test | Signed-Rank |
| Category (Label_{—} | Mean | p-value | p-value |
| Morphology (Asymmetry) | 1.5 | <.0001 | 0.0002 |
| Morphology (Imbalance) | 1.2 | <.0001 | 0.0002 |
| Skin Quality (Pigmentation) | 1.4 | <.0001 | 0.0001 |
| Skin Quality (Scars) | 0.9 | <.0001 | 0.0005 |
| Skin Quality (Vasculature) | 1.6 | <.0001 | 0.0002 |
| Skin Quality (Visible Pores) | 1.4 | <.0001 | 0.0001 |
| Skin (Dehydration) | 1.6 | <.0001 | 0.0001 |
| Skin (Loss of Elasticity) | 1.3 | <.0001 | 0.0001 |
| Skin (Surface Roughness) | 1.9 | <.0001 | 0.0001 |
| Volume (Sagging) | 1.3 | <.0001 | 0.0002 |
| Volume (Volume Loss) | 1.2 | <.0001 | 0.0002 |
| Wrinkles (Dynamic Wrinkles) | 1.1 | <.0001 | 0.0002 |
| Wrinkles (Static Wrinkles) | 1.3 | <.0001 | 0.0001 |

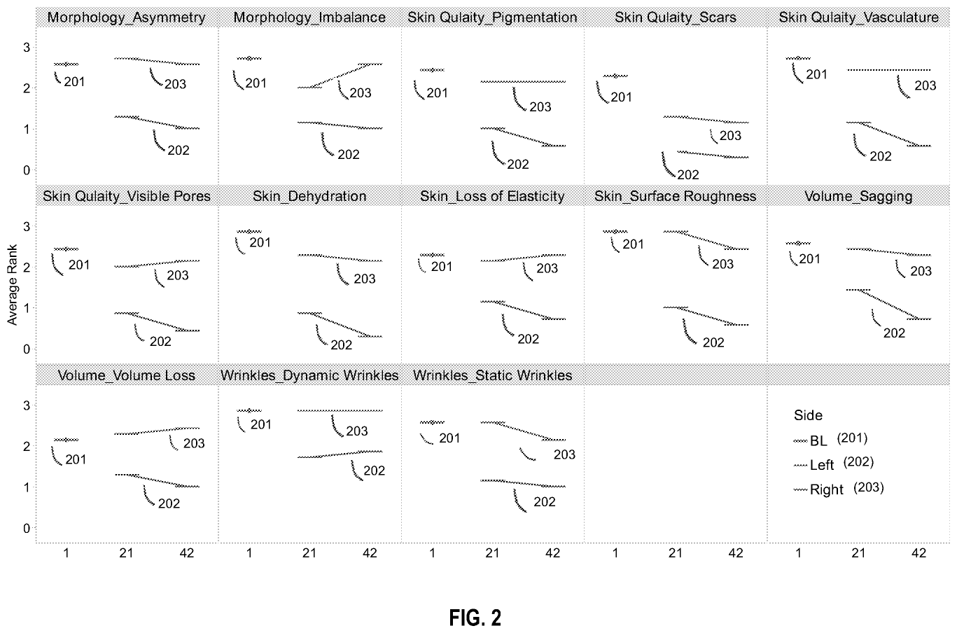

Mean GRS scores with standard errors are shown in FIG. 2 for baseline and each follow-up visit for both the treatment side and placebo side. Mean baseline scores were reflective of mild to moderate severity, with the most severe average scores observed for loss of elasticity, surface roughness, vasculature, dehydration, visible pores, and imbalance, as well as pigmentation. For each domain, the improvement on the treated (left) side was apparent by day 21, and additional improvement was observed at week 6 (day 42). The non-treated sides (right) tended to remain unchanged from baseline.

While an improvement was observed for each GRS domain, including those for which one would not expect a topical treatment to have an effect (such as volume loss), assessment of the degree of improvement in each domain reveals that the greatest changes (from day 1 to day 42) were present in those domains consistent with topical treatments (FIG. 2). Domains in which a >1-point improvement was observed include dehydration, visible pores, static wrinkles, surface roughness, pigmentation, and vasculature. While “imbalance” is considered a morphologic domain, and refers to the profile (“asymmetry” refers to the frontal view of the face), this term may be somewhat unclear to subjects, and the degree of change observed in this domain, as well as the larger confidence interval, may be reflective of some patients assessing balanced skin tone rather than balance of facial anatomic structures, or may be reflective of patient 'assessing the magnitude of aesthetic skin changes experienced between the treatment and placebo sides of the face due to the split-face study design.

The blinded reviewer correctly identified the treatment side in 100% of subjects across all domains evaluated (surface roughness, static wrinkles, vasculature, visible pore size, pigmentation, and scars).

### DISCUSSION

The GLPSGLT topical is a unique and novel topical formulation that includes ingredients known to stimulate collagen and elastin production in the skin, increase skin cell turnover, and increase keratinocyte proliferation. It is believed this represents the first study of its kind to evaluate such a combination of ingredients for both cosmetic enhancement and the reversal of drug-induced skin changes related to GLP-1 RA medications, specifically tested on GLP-1 RA treated subjects. This study provides strong evidence, in a small number of patients, that this combination is effective for improving skin elasticity, surface roughness, skin texture and smoothness, dehydration, appearance of pores, fine lines and wrinkles, and pigmentation. These improvements were consistently captured across three different measures: the GRS scale, patient satisfaction surveys, and independent physician review. Increased skin elasticity and firmness at day 42 on the treated side—observed in all of patients—presents an opportunity for future more in-depth investigation related to histopathological changes to the skin. It is theorized that the GLPSGLT topical product increased epidermal thickness, in direct contrast to observed epidermal thinning in GLP-1 RA treated patients; however, skin biopsies and histopathological analysis will be required to definitively conclude GLPSGLT topical's effects to skin structure.

While a full review of the body of evidence behind each ingredient in GLPSGLT Topical not presented herein, the rationale behind the main active ingredients is given below.

Carotenoid byproducts are a well-known and well-established ingredient in topical formulations, typically prescribed to address signs of aging. In previous clinical study using the GRS as a primary endpoint on 10 [n=10] subjects, a topical product using a proprietary complex form of vitamin A as a primary active ingredient led to clinically significant improvements in 13 of 13 domains. Notably, 95% of subjects showed improvement to static wrinkles, and 95% demonstrated improvement to surface roughness [17]. Additionally, though very few of the patients in the referenced study presented with severe elasticity loss at baseline, 100% experienced improvement in their skin elasticity at the conclusion of the study [17]. These findings demonstrated that vitamin A as a primary active ingredient showed significant promise for addressing GLP-1 RA induced accelerated signs of aging and loss of elasticity. This premise led to the formulation of a bioactive soluble vitamin A derivative in a novel peptide matrix for enhanced dermal availability and decreased negative inflammation.

Retinoids also improve several aspects of skin health and function thought to be negatively influenced by GLP-1 RA use. Vitamin A acts on retinoic acid receptors (RARs) and retinoid X receptors (RXRs) in keratinocytes, which promotes cell renewal and keratinocyte proliferation through nuclear receptor mediated gene transcription [18]. Specifically, vitamin A increases proliferation of keratinocytes in the epidermis, leading to epidermal thickening while GLP-1 RAs suppress keratinocyte proliferation and lead to epidermal thinning [19][20][4]. Retinoids influence keratinocyte differentiation, stimulating keratinocytes to develop properly into cells needed for proper skin barrier function. HaCaT or spontaneous immortalized human keratinocyte line evaluation with GLP-1 use impairs keratinocyte inflammatory signal by activating AMP-activated protein kinase (AMPK) and restraining macrophage migration, thus impairing apoptosis and the natural pathway of keratinocyte repair [6]. In addition, transforming growth factor beta 1 behavior is modified in ways that appear to overlap what is seen in glucocorticoid therapy[21]. With continual use, retinoids are known to strengthen the protective function of the epidermis, restrain transepidermal water loss, and protect collagen against degradation [18]. As GLP-1 RAs are thought to decrease skin tensile strength & barrier function by thinning the epidermis—and reduce skin elasticity due to decreased barrier function leading to collagen and elastin degradation—bioavailable peptide and vitamin A derivatives lead to promotion of a healthy skin barrier and protective effects against collagen and elastin degradation, allowing it to act as a primary active in the GLPSGLT formula.

Additional actives in the GLPSGLT formula include palmitoyl oligopeptide and palmitoyl tetrapeptide. Peptides are short chains of amino acids and are the building blocks of collagen and elastin. Both palmitoyl oligopeptide and palmitoyl tetrapeptide have been demonstrated to improve skin elasticity, as well as the appearance of fine lines and wrinkles, when used in combination topical formulations [22]. Palmitoyl oligopeptide and palmitoyl tetrapeptide provide demonstrable evidence of elasticity and aesthetic improvements counter to GLP-1 RA's elasticity-reducing and accelerative effects on aging.

Strengths of this study include excellent retention as well as the inclusion of a wide range of patient skin types in a relatively small sample size (n=7) which makes for more informative study results. The use of a split-face design allowed for direct comparison between study topical and placebo treated skin, allowing for more conclusive analysis of GLPSGLT topical's effects. Also positive was GLPSGLT topical's limited side effect profile, indicating broad tolerance in a cohort of patients with compromised skin barrier strength. The results of this study indicate that the novel GLPSGLT topical formulation may help reverse skin changes associated with GLP-1 and SGLT-2 use, including reduced elasticity, surface roughness, and impaired hydration. However, without timely intervention, these structural and functional skin changes may become long-standing or potentially permanent, reinforcing the need for early treatment and preventative care in patients using these medications.

Limitations include a small cohort of patients (n=7). While the inclusion of blinded third party photographic review and a patient satisfaction survey with questions overlapping with the domains of the GRS assessment increase the rigor of the study beyond what would be achievable with the GRS alone. The authors recognize that these assessments are subjective, and like all subjective measures, may be prone to individual interpretation and bias. Inclusion of an objective scale or outcome measure would strengthen study findings.

Furthermore, this study would have benefited from histopathological skin analysis to assess structural skin changes in GLP-1 analog and SGLT analog treated subjects prior to and after treatment with GLPSGLT topical. In future study, histopathological analysis should be employed to make more informed conclusions about structural skin changes, including the presence of collagen bundles in the skin before and after treatment. This study would have also benefited from expanding the inclusion criteria to corticosteroid users experiencing skin atrophy. Given the potential keratinocyte suppressing mechanisms described for GLP-1 RAs, and the keratinocyte stimulating effects of GLPSGLT Topical, this study would have benefited from additionally examining patients with corticosteroid skin thinning: a more well-established cause of keratinocyte-suppression-related skin atrophy. Last, the safety and tolerability described here are short-term (42 days) and do not necessarily reflect long term safety.

While the current study focuses on the adverse dermatologic effects of GLP-1 RAs, it is important to note their emerging role in modulating inflammation. GLP-1s have demonstrated anti-inflammatory actions in both in vitro and in vivo models, suggesting potential therapeutic utility in conditions such as psoriasis, keloid scarring, and other chronic inflammatory skin diseases. To fully understand the dual nature of GLP-is-both their beneficial and adverse effects-future studies should incorporate dermatopathological assessments and long-term clinical follow-ups across a range of inflammatory and structural skin disorders.

GLPSGLT was formulated specifically to address the growing concern for skin changes observed in GLP-1 analog and SGLT analog treated patients. The GLPSGLT topical

This study supports a role for this formulation as a supplementary at-home treatment for both GLP analog and SGLT users to address related losses of elasticity and firmness, in addition to a wide range of other age-related aesthetic concerns across multiple skin types.

Based on the evidence gathered in this pilot study, GLPSGLT topical is a promising and well-tolerated topical serum for an array of skin quality issues. In GLP-1 analog and SGLT analog treated patients, there is a particular concern for loss of elasticity, volume loss, and accelerated signs of aging. In particular for surface roughness, loss of elasticity, volume loss, wrinkles (both static and dynamic), and skin hydration, the formulation is associated with improvement and a high degree of patient satisfaction. In addition, observed improvements in visible pore size, scar appearance, vasculature, and pigmentation suggest a possible effect on skin quality. Taken together, these data support additional study of GLPSGLT topical for slowing age and GLP-1 RA-related changes to the skin structure as well as facial skin rejuvenation.

As the popularity of GLP-1 and SGLT-2 medications increases, particularly among individuals seeking to lose only 5-10 pounds, it is important to emphasize the need for caution in off-label use. These medications are designed and approved for the treatment of obesity and type 2 diabetes; use beyond these indications should involve a thorough consideration of risks, including potential long-term impacts to skin health and integrity. Patients and providers should weigh the benefits of minor weight loss against the possible dermatologic side effects that may not be immediately reversible.

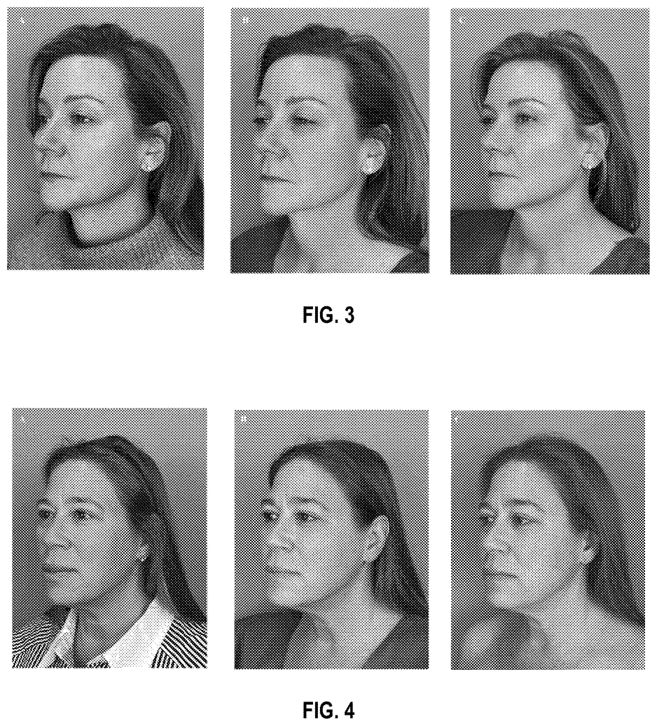

FIG. 3 shows photographs of a 54 year old female with a present history of GLP-1 therapy for 6 months and stable weight demonstrating the treatment side-left-of the face at baseline, at 21 days, and at 42 days post topical application of GLPSGLT serum twice daily. Note that surface roughness, static wrinkles, visible pores, pigmentation and vasculature were rated as statistically significant improvement compared to placebo control by subject, principle investigator and independent—blinded to treatment-plastic surgeon.

FIG. 4 shows photographs of a 51 year-old female on GLP-1 therapy for 14 months and stable weight presenting baseline for left treatment side, after 21 days of twice daily application of GLPSGLT Serum; and after 42 days of twice daily application of GLPSGLT Serum.

FIG. 5 shows photographs of a 63 year old female on GLP-1 therapy for 1 year and stable weight at baseline with skin characterized by prominent fine lines & redness; and after 42 days of twice daily application of GLPSGLT Serum.

As used in the specification and appended claims, and in addition to their ordinary meanings, the terms “substantial” or “substantially” mean to within acceptable limits or degree to one having ordinary skill in the art. For example, “substantially free of” a material means that one skilled in the art considers the remaining amount of material to be acceptable.

As used in the specification and the appended claims and in addition to its ordinary meaning, the terms “approximately” and “about” mean to within an acceptable limit or amount to one having ordinary skill in the art. The term “about” generally refers to plus or minus 15% of the indicated number. For example, “about 10” may indicate a range of 8.5 to 11.5. For example, “approximately the same” means that one of ordinary skill in the art considers the items being compared to be the same.

In the present disclosure, when percentages are used to identify the amount of a component, the percentages are based on the weight of the component over the total weight of the composition (unless the context indicates another basis of calculating the percentage). When a component is provided in a mixture (such as in a mixture comprising a liquid medium), the weight of the component itself (not including the liquid medium or other components in the mixture) is used to calculate its percentage in the composition.

As used in the specification and appended claims, the terms “a,” “an,” and “the” include both singular and plural referents, unless the context clearly dictates otherwise. Thus, for example, “a retinoid” includes one retinoid and multiple retinoids.

In the present disclosure, numeric ranges are inclusive of the numbers defining the range. It should be recognized that chemical structures and formula may be elongated or enlarged for illustrative purposes.

As disclosed herein, a number of ranges of values are provided. Each smaller range between any stated value or intervening value in a stated range and any other stated or intervening value in that stated range is encompassed within the invention. The upper and lower limits of these smaller ranges may independently be included or excluded in the range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present teachings, some exemplary methods and materials are now described.

All patents and publications referred to herein are expressly incorporated by reference.

### Exemplary Embodiments

Exemplary embodiments provided in accordance with the presently disclosed subject matter include, but are not limited to, those set forth in the claims, description, and examples.

Embodiment 1. A method of improving one or more facial aesthetics of a subject, comprising: identifying a subject receiving or about to receive a therapeutic having an adverse effect on one or more facial aesthetics; and applying to facial skin of the identified subject a topical composition comprising an agent that reverses or ameliorates the adverse effect of the therapeutic on said facial aesthetics.

Embodiment 2. The method of embodiment 1, wherein the therapeutic is selected from GLP-1 agonists, SGLT-2 inhibitors, corticosteroids, or any drug known to decrease collagen and elastin in the dermis.

Embodiment 3. The method of embodiment 1, wherein the therapeutic is a GLP-1 receptor agonist.

Embodiment 4. The method of embodiment 1, wherein the therapeutic is a SGLT-2 inhibitor.

Embodiment 5. The method of embodiment 1, wherein the therapeutic is a corticosteroid.

Embodiment 6. The method of embodiment 5, wherein the corticosteroid is a glucocorticoid.

Embodiment 7. The method of embodiment 1, wherein the topical composition stimulates keratinocytes.

Embodiment 8. The method of any of embodiments 1 to 7, wherein the topical composition comprises the agent in an amount effective to stimulate keratinocytes.

Embodiment 9. The method of any of embodiments 1 to 8, wherein the topical composition induces differentiation of keratinocytes.

Embodiment 10. The method of any of embodiments 1 to 9, wherein the topical composition increases production of collagen and/or elastin.

Embodiment 11. The method of any of embodiments 1 to 10, wherein the topical composition comprises the agent in an amount effective to counteract impairment of keratinocyte inflammatory signaling by the therapeutic.

Embodiment 12. The method of any of embodiments 1 to 11, wherein the topical composition further comprises one or more of an aliphatic oligopeptide and hyaluronic acid.

Embodiment 13. The method of any of embodiments 1 to 12, wherein the composition is applied at least once per day for at least 21 days.

Embodiment 14. The method of any of embodiments 1 to 12, wherein the composition is applied at least once per day for at least 42 days.

Embodiment 15. The method of any of embodiments 1 to 14, wherein the composition is a serum applied to the facial skin without removal.

Embodiment 16. The method of any of embodiments 1 to 15, further comprising treating the facial skin of the subject by a dermatological procedure before, during or after applying the topical composition.

Embodiment 17. The method of embodiment 16, further comprising wherein the dermatological procedure is selected from the group consisting of microdermabrasion, hydradermabrasion, dermabrasion, dermal planing, and combinations thereof.

Embodiment 18. The method of embodiment 16, wherein the dermatological procedure comprises abrading a surface of facial skin of a subject to remove dead cells and/or cell debris from a surface of the facial skin; wherein said abrading of the facial skin is performed before or during said applying the topical composition to the facial skin.

Embodiment 19. The method of embodiment 18, wherein said abrading is by microdermabrasion or by hydradermabrasion.

Embodiment 20. The method of embodiment 16, wherein the topical composition is applied to the facial skin by injection or microinjection.

Embodiment 21. The method of embodiment 16, wherein the topical composition is applied to the facial skin by infusion.

Embodiment 22. A topical composition comprising: a retinoid; an aliphatic oligopeptide; hyaluronic acid; and an aqueous medium.

Embodiment 23. The composition of embodiment 22, wherein the retinoid is selected from the group consisting of retinol, retinal, retinyl esters, and mixtures thereof.

Embodiment 24. The composition of embodiment 22, wherein the retinoid comprises Vitamin A.

Embodiment 25. The composition of embodiment 22 or 24, wherein the retinoid comprises a retinyl ester.

Embodiment 26. The composition of embodiment 22 or 24, wherein the retinoid comprises a retinyl palmitate.

Embodiment 27. The composition of any of embodiments 22 to 26, wherein the aliphatic oligopeptide is palmitoyl oligopeptide.

Embodiment 28. The composition of any of embodiments 22 to 27, wherein the aliphatic oligopeptide is palmitoyl tetrapeptide.

Embodiment 29. A method of improving one or more facial aesthetics of a subject, comprising: identifying a subject receiving or about to receive a therapeutic having an adverse effect on one or more facial aesthetics; and applying to facial skin of the identified subject a topical composition according to any of embodiments 22 to 28.

It will be understood that various aspects or details of the invention may be changed without departing from the scope of the invention. Furthermore, the foregoing description is for the purpose of illustration only, and not for the purpose of limitation—the invention being defined by the embodiments.

### REFERENCES

### Citations

1. Holst J J (2007) The Physiology of Glucagon-like Peptide 1. Physiological Reviews 87: 1409-1439
2. Filippatos T D, Panagiotopoulou T V, Elisaf M S (2014) Adverse Effects of GLP-1 Receptor Agonists. Rev Diabet Stud 11: 202-230
3. Ridha Z, Fabi S G, Zubar R, Dayan S H (2024) Decoding the Implications of Glucagon-like Peptide-1 Receptor Agonists on Accelerated Facial and Skin Aging. Aesthetic Surgery Journal 44: NP809-NP818
4. Xu X, Lin L, Chen P, Yu Y, Chen S, Chen X, Shao Z (2019) Treatment with liraglutide, a glucagon-like peptide-1 analogue, improves effectively the skin lesions of psoriasis patients with type 2 diabetes: A prospective cohort study. Diabetes Res Clin Pract 150: 167-173
5. Nagae K, Uchi H, Morino-Koga S, Tanaka Y, Oda M, Furue M (2018) Glucagon-like peptide-1 analogue liraglutide facilitates wound healing by activating PI3K/Akt pathway in keratinocytes. Diabetes Res Clin Pract 146: 155-161
6. Yang J, Wang Z, Zhang X (2019) GLP-1 receptor agonist impairs keratinocytes inflammatory signals by activating AMPK. Exp Mol Pathol 107: 124-128
7. de Jong E M, Ferrier C M, de Zwart A, Wauben-Penris P J, Korstanje C, van de Kerkhof PC (1995) Effects of topical treatment with budesonide on parameters for epidermal proliferation, keratinization and inflammation in psoriasis. J Dermatol Sci 9: 185-194
8. Afifi T, de Gannes G, Huang C, Zhou Y (2005) Topical therapies for psoriasis: evidence-based review. Can Fam Physician 51: 519-525
9. Stojadinovic O, Lee B, Vouthounis C, Vukelic S, Pastar I, Blumenberg M, Brem H, Tomic-Canic M (2007) Novel genomic effects of glucocorticoids in epidermal keratinocytes: inhibition of apoptosis, interferon-gamma pathway, and wound healing along with promotion of terminal differentiation. J Biol Chem 282: 4021-4034
10. Lange K, Kleuser B, Gysler A, Bader M, Maia C, Scheidereit C, Korting H C, Schafer-Korting M (2000) Cutaneous Inflammation and Proliferation in vitro: Differential Effects and Mode of Action of Topical Glucocorticoids. Skin Pharmacology and Applied Skin Physiology 13: 93-103
11. Kimura T, Doi K (1999) Dorsal skin reactions of hairless dogs to topical treatment with corticosteroids. Toxicol Pathol 27: 528-535
12. Kolbe L, Kligman A M, Schreiner V, Stoudemayer T (2001) Corticosteroid-induced atrophy and barrier impairment measured by non-invasive methods in human skin. Skin Res Technol 7: 73-77
13. Shields B A, Nakakura A M (2023) Nutrition Considerations for Burn Patients: Optimizing Recovery and Healing. Eur Burn J 4: 537-547
14. Wicke C, Halliday B, Allen D, Roche N S, Scheuenstuhl H, Spencer M M, Roberts AB, Hunt TK (2000) Effects of steroids and retinoids on wound healing. Arch Surg 135: 1265-1270
15. Jain R, Huang P, Ferraz R M (2017) A new tool to improve delivery of patient-engaged care and satisfaction in facial treatments: the Aesthetic Global Ranking Scale. J Cosmet Dermatol 16: 132-143
16. Few J, Lee M, Semersky A, Mariscal E, Pryor S, Talati A (2023) A Single-Center Study Evaluating the Effects of a Topical Serum Combining Postbiotics, Peptides, and Botanical Extracts on Skin. Aesthetic Surgery Journal Open Forum 5
17. Few J, Lee M J, Semersky A, Mariscal E, Vachon G (2022) A Single-Center Study Evaluating the Effects of a Novel Retinol and Cannabidiol Combination Topical on Facial Skin. Aesthetic Surgery Journal Open Forum 4
18. Zasada M, Budzisz E (2019) Retinoids: active molecules influencing skin structure formation in cosmetic and dermatological treatments. Postepy Dermatol Alergol 36: 392-397
19. Chapellier B, Mark M, Messaddeq N, Calleja C, Warot X, Brocard J, Gerard C, Li M, Metzger D, Ghyselinck N B, Chambon P (2002) Physiological and retinoid-induced proliferations of epidermis basal keratinocytes are differently controlled. Embo j 21: 3402-3413
20. VanBuren C A, Everts H B (2022) Vitamin A in Skin and Hair: An Update. Nutrients 14: 2952
21. Nincevic V, Zjalic M, Kolaric T O, Smolic M, Kizivat T, Kuna L, Vcev A, Tabll A, Curcic IB (2022) Renoprotective Effect of Liraglutide Is Mediated via the Inhibition of TGF-Beta 1 in an LLC-PK1 Cell Model of Diabetic Nephropathy. Curr Issues Mol Biol 44: 1087-1114
22. Hahn H J, Jung H J, Schrammek-Drusios M C, Lee S N, Kim J H, Kwon S B, An I S, An S, Ahn KJ (2016) Instrumental evaluation of anti-aging effects of cosmetic formulations containing palmitoyl peptides, Silybum marianum seed oil, vitamin E and other functional ingredients on aged human skin. Exp Ther Med 12: 1171-1176

What is claimed is:

1. A method of improving one or more facial aesthetics of a subject, comprising:
identifying a subject receiving or about to receive a therapeutic having an adverse effect on one or more facial aesthetics; and
applying to facial skin of the identified subject a topical composition comprising an agent that reverses or ameliorates the adverse effect of the therapeutic on said facial aesthetics.

2. The method of claim 1, wherein the therapeutic is selected from GLP-1 agonists, SGLT-2 inhibitors, corticosteroids, or any drug known to decrease collagen and elastin in the dermis.

3. The method of claim 1, wherein the topical composition further comprises one or more of an aliphatic oligopeptide and hyaluronic acid.

4. The method of claim 1, wherein the composition is applied at least once per day for at least 21 days.

5. The method of claim 1, wherein the composition is applied at least once per day for at least 42 days.

6. The method of claim 1, wherein the composition is a serum applied to the facial skin without removal.

7. The method of claim 1, further comprising
treating the facial skin of the subject by a dermatological procedure before, during or after applying the topical composition.

8. The method of claim 7, further comprising wherein the dermatological procedure is selected from the group consisting of microdermabrasion, hydradermabrasion, dermabrasion, dermal planing, and combinations thereof.

9. The method of claim 7, wherein the dermatological procedure comprises abrading a surface of facial skin of a subject to remove dead cells and/or cell debris from a surface of the facial skin;
wherein said abrading of the facial skin is performed before or during said applying the topical composition to the facial skin.

10. The method of claim 9, wherein said abrading is by microdermabrasion or by hydradermabrasion.

11. The method of claim 7, wherein the topical composition is applied to the facial skin by injection or microinjection.

12. The method of claim 7, wherein the topical composition is applied to the facial skin by infusion.

13. A topical composition comprising:
a retinoid;
an aliphatic oligopeptide;
hyaluronic acid; and
an aqueous medium.

14. The composition of claim 13, wherein the retinoid is selected from the group consisting of retinol, retinal, retinyl esters, and mixtures thereof.

15. The composition of claim 13, wherein the retinoid comprises Vitamin A.

16. The composition of claim 13, wherein the retinoid comprises a retinyl ester.

17. The composition of claim 13, wherein the retinoid comprises a retinyl palmitate.

18. The composition of claim 13, wherein the aliphatic oligopeptide is palmitoyl oligopeptide.

19. The composition of claim 13, wherein the aliphatic oligopeptide is palmitoyl tetrapeptide.

20. A method of improving one or more facial aesthetics of a subject, comprising:
identifying a subject receiving or about to receive a therapeutic having an adverse effect on one or more facial aesthetics; and
applying to facial skin of the identified subject a topical composition according to claim 13.
